(19) United States
(12) Patent Application Publication
(10) Pub. No.: US 2019/0388971 A1
(43) Pub. Date: Dec. 26, 2019

(54) ONE-STEP MANUFACTURING METHOD OF LAMINATED MOLDING POROUS COMPONENT

(71) Applicant: KOREA INSTITUTE OF INDUSTRIAL TECHNOLOGY, Cheonan-si (KR)

(72) Inventors: Kyung Hwan JUNG, Daejeon (KR); Gun Hee KIM, Incheon (KR); Byoung Soo LEE, Gangneung-si (KR); Hyung Giun KIM, Gangneung-si (KR); Seung Min YANG, Gangneung-si (KR); Kang Min KIM, Seoul (KR); Won Rae KIM, Gangneung-si (KR); Min Ji HAM, Gangwon-do (KR); Kyung Hoon KIM, Daejeon (KR); Chang Woo LEE, Seoul (KR)

(73) Assignee: KOREA INSTITUTE OF INDUSTRIAL TECHNOLOGY, Cheonan-si (KR)

(21) Appl. No.: 16/158,512

(22) Filed: Oct. 12, 2018

(30) Foreign Application Priority Data
Jun. 20, 2018 (KR) 10-2018-0070823

(51) Int. Cl. B22F 3/11; B22F 3/105; A61L 27/56; A61F 2/30; A61L 27/06

(52) U.S. Cl. CPC B22F 3/11; B22F 3/105; A61L 27/56; B33Y 10/00; A61L 27/06; A61F 2002/30985; A61F 2310/00023; A61F 2/30771

(57) ABSTRACT

An exemplary embodiment provides a method of manufacturing a porous component having a base material layer and a porous layer through one-step laminated-molding, whereby it is possible to provide a manufacturing time when manufacturing a product and to provide a porous component in which the shape and size of a porous layer can be controlled. An implant including the porous component has an increased bone contact ratio, so bone growth between bones can be improved and products fitting to the frames of patients can be easily designed.

### BACKGROUND OF THE INVENTION

### Field of the Invention

The present invention relates to a one-step manufacturing method of laminated molding porous component and, more particularly, to a method of manufacturing a porous component having a base material layer and a porous layer through one step using a laminated molding technology to a process of manufacturing a porous component for increasing a bone contact ratio of an implant.

### Description of the Related Art

An implant means a material that is used when reconstructing a shape or substituting for a function by implanting an artificial material or a natural material in a lost portion to compensate for a loss of a biological tissue. In general, an implant means a biological material for substituting for hard tissues of a human body in dentistry or orthopedics, and studies related to dental implants have been actively conducted since the mid-1960s.

Metallic materials having high strength and hardness and low biological toxicity are selected as the materials of implants. In particular, titanium and titanium alloys, which are materials having excellent biocompatibility, have been known as having not only good biocompatibility for surrounding tissues, but large resistance against corrosion and little biological toxicity. For this reason, in the early stage of the study related to implants, titanium or titanium alloys were used as implants through simple machining.

An implant can be implanted to a lost portion only when it has compatibility to an existing biological tissue, so most implants are coated with a biological tissue adhesive on the surfaces. In particular, bone cement that is an adhesive inducing quick regeneration of a bone tissue has been used for complex fracture restoration and artificial joint operations that frequently occur due to traffic accidents etc. in the field of orthopedics and for dentin restoration of non-regenerative teeth in dentistry.

However, bioactive substances coated on the surfaces are dissolved too fast, and high temperature is generated in the coating process which makes it difficult to expect the effect of coated materials. Further, it has been reported that substances coming off coating layers may interfere with bonding of bones or may cause side effects such as inflammation.

In order to solve this problem, there has been proposed a method of coating an implant with a porous structure on the surface to improve growth of bones even without cement, and products using this method have been released.

However, this method also has a problem with bonding between an implant and a porous structure, and it is required to add a process of manufacturing a separate porous structure and then attaching it to an implant, which reduces productivity and increases the manufacturing costs of implants.

3D printing that has been recently actively studied may be an alternative measure that can solve the problem. It is possible to laminated-mold metallic materials such as titanium that is generally used as the material of implants, using 3D printing, so it may be possible to develop a new implant using this method.

### SUMMARY OF THE INVENTION

In order to solve the problems, an object of the present invention is to provide a method of manufacturing a porous component having a base material layer and a porous layer through a one-step laminated molding.

Another object of the present invention is to provide a method of reducing a process time and controlling the shape and size of a porous layer when manufacturing a product including a porous component.

The technical object to implement in the present invention are not limited to the technical problems described above, and other technical objects that are not stated herein will be clearly understood by those skilled in the art from the following specifications.

In order to achieve the objects, an embodiment of the present invention provides a one-step manufacturing method of a laminated molding porous component, the method including the steps of: layering metallic particles; forming a base material layer by repeatedly melting and cooling the metallic particles by radiating a laser to the layered metallic particles; forming a first porous layer by radiating a laser while adjusting a hatch distance and a point distance to form laser radiation points having a predetermined diameter D on the base material layer; layering metallic particles, which are the same as the metallic particles, on the first porous layer; and forming a second porous layer by radiating a laser while adjusting a hatch distance and a point distance to form laser radiation points having a predetermined diameter D on the first porous layer.

In an embodiment of the present invention, the metallic particles may be one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

In an embodiment of the present invention, the laser may have energy equal to or greater than complete melting energy of the metallic particles in the step of forming a base material layer and in the step of forming a first porous layer.

In an embodiment of the present invention, the hatch distance and the point distance may be greater than the diameter D of the laser radiation points in the step of forming a first porous layer and in the step of forming a second porous layer.

In an embodiment of the present invention, the diameter D of the laser radiation points may be in proportion to source power and exposure time of the laser and the exposure time may be in inverse proportion to the scan speed of the laser.

In an embodiment of the present invention, the source power of the laser may be 50 W to 1 KW, and the scan speed may be 0.1 m/s to 8 m/s.

In an embodiment of the present invention, the hatch distance and the point distance may be 100 to 1000 μm, respectively.

In an embodiment of the present invention, the first porous layer may be engraved.

In order to achieve the objects, an embodiment of the present invention provides a one-step manufacturing method of a laminated molding porous component, the method including the steps of: layering metallic particles; forming a base material layer by repeatedly melting and cooling the metallic particles by radiating a laser to the layered metallic particles; layering metallic particles, which are the same as the metallic particles, on the base material layer; forming a first porous layer by radiating a laser while adjusting a hatch distance and a point distance to form laser radiation points having a predetermined diameter D on the metallic particles layered on the base material layer; layering metallic particles, which are the same as the metallic particles, on the first porous layer; and forming a second porous layer by radiating a laser while adjusting a hatch distance and a point distance to form laser radiation points having a predetermined diameter D on the metallic particles layered on the first porous layer.

In another embodiment of the present invention, the metallic particles may be one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

In another embodiment of the present invention, the laser may have energy equal to or greater than complete melting energy of the metallic particles in the step of forming a base material layer.

In another embodiment of the present invention, in the step of forming a first porous layer and in the step of forming a second porous layer, the laser may have energy equal to or greater than 0.2 times the complete melting energy within a range equal to or less than the complete melting energy of the metallic particles.

In another embodiment of the present invention, the hatch distance and the point distance may be greater than the diameter D of the laser radiation points in the forming of a first porous layer and in the forming of a second porous layer.

In another embodiment of the present invention, the diameter D of the laser radiation points may be in proportion to source power and exposure time of the laser and the exposure time may be in inverse proportion to the scan speed of the laser.

In another embodiment of the present invention, the source power of the laser may be 50 W to 1 KW and the scan speed may be 0.1 m/s to 8 m/s.

In another embodiment of the present invention, the hatch distance and the point distance may be 100 to 1000 μm, respectively.

In another embodiment of the present invention, the first porous layer may be embossed.

In another embodiment of the present invention, the laser radiation points in the step of forming a second porous layer may be arranged not to overlap the laser radiation points on the first porous layer.

In order to achieve the objects, another embodiment of the present invention provides a laminated-molding porous component formed by the method.

In order to achieve the objects, another embodiment of the present invention provides an implant having an increased bone contact ratio and including the porous component.

### BRIEF DESCRIPTION OF THE DRAWINGS

FIG. 1 is a flowchart showing a one-step manufacturing method of laminated molding porous component according to an embodiment of the present invention;

FIG. 2 is a picture showing a laser radiation method when forming a base material layer according to the present invention;

FIG. 3 is a picture showing a laser radiation method when forming a porous layer according to the present invention;

FIG. 4 is a picture showing a hatch distance and a point distance of laser radiation points according to the present invention;

FIG. 5 is a schematic view showing a one-step manufacturing method of laminated molding porous component;

FIG. 6 is a flowchart showing a one-step manufacturing method or laminated molding porous component according to another embodiment of the present invention;

FIG. 7 is a schematic view showing a one-step manufacturing method of laminated molding porous component according to another embodiment of the present invention;

FIG. 8 is a schematic view vertically showing laser radiation points according to the present invention; and

FIG. 9 is a picture of the surface of a porous layer according to an embodiment of the present invention.

### DETAILED DESCRIPTION OF THE PREFERRED EMBODIMENTS

Hereinafter, the present invention is described with reference to the accompanying drawings. However, the present invention may be modified in various different ways and is not limited to the embodiments described herein. Further, in the accompanying drawings, components irrelevant to the description will be omitted in order to obviously describe the present invention, and similar reference numerals will be used to describe similar components throughout the specification.

Throughout the specification, when an element is referred to as being “connected with (coupled to, combined with, in contact with)” another element, it may be “directly connected” to the other element and may also be “indirectly connected” to the other element with another element intervening therebetween. Further, unless explicitly described otherwise, “comprising” any components will be understood to imply the inclusion of other components rather than the exclusion of any other components.

Terms used in this specification are used only in order to describe specific exemplary embodiments rather than limiting the present invention. Singular forms are intended to include plural forms unless the context clearly indicates otherwise. It will be further understood that the terms “comprise” or “have” used in this specification, specify the presence of stated features, numerals, steps, operations, components, parts, or a combination thereof, but do not preclude the presence or addition of one or more other features, numerals, steps, operations, components, parts, or a combination thereof.

Hereinafter, embodiments of the present invention are described in detail with reference to the accompanying drawings.

A one-step manufacturing method of laminated molding porous component is described hereafter.

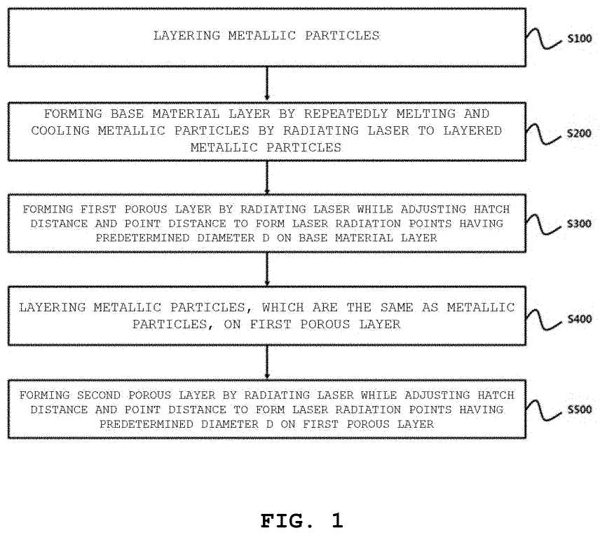

Referring to FIG. 1, an embodiment of the present invention provides a one-step manufacturing method of a laminated molding porous component, the method including the steps of: layering metallic particles (S100); forming a base material layer by repeatedly melting and cooling the metallic particles by radiating a laser to the layered metallic particles (S200); forming a first porous layer by radiating a laser while adjusting a hatch distance and a point distance to form laser radiation points having a predetermined diameter D on the base material layer (S300); layering metallic particles, which are the same as the metallic particles, on the first porous layer (S400); and forming a second porous layer by radiating a laser while adjusting a hatch distance and a point distance to form laser radiation points having a predetermined diameter D on the first porous layer (S500).

The metallic particles may be one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

In particular, titanium and titanium-based alloys, which are materials having excellent biocompatibility, have been known as having not only good biocompatibility for surrounding tissues, but large resistance against corrosion and little biological toxicity, so they are preferable. However, the present invention is not limited thereto and the metallic particles described above can be selectively used.

The laser may have energy equal to or greater than complete melting energy of the metallic particles in the step of forming the base material layer and the step of forming the first porous layer.

In the steps of forming the second porous layer, the laser may have energy equal to or greater than 0.2 times the complete melting energy within a range equal to or less than the complete melting energy of the metallic particles.

When energy greater than the complete melting energy is applied to the metallic particles, the metallic particles may be completely melted and densified. When smaller energy is applied to the metallic particles, the metallic particles may be formed in a porous type without being densified.

That is, when forming the base material layers and the second porous layer in the present invention, the base material layers can be densified by inputting energy equal to or greater than the complete melting energy and the second porous layer can be formed in porous type by inputting energy equal to or greater than 0.2 times the complete melting energy within a range equal to or less than the complete melting energy. The porosity is another factor that forms a porous structure separate from radiating a laser while adjusting a hatch distance and a point distance when forming laser radiation points. When the laser has energy less than 0.2 times the complete melting energy of the metallic particles, the metallic particles are never melted, so it is not preferable.

The forming of a first porous layer forms a porous layer and inputs energy equal to or greater than the complete melting energy of the metallic particles. This is because the first porous layer is formed by radiating a laser to a base material layer and the base material layer is already densified, so energy equal to or greater than complete melting energy is required to from a porous structure by melting the layers again.

The hatch distance and the point distance may be greater than the diameter D of the laser radiation points in the step of forming the first porous layer and the step of forming the second porous layer.

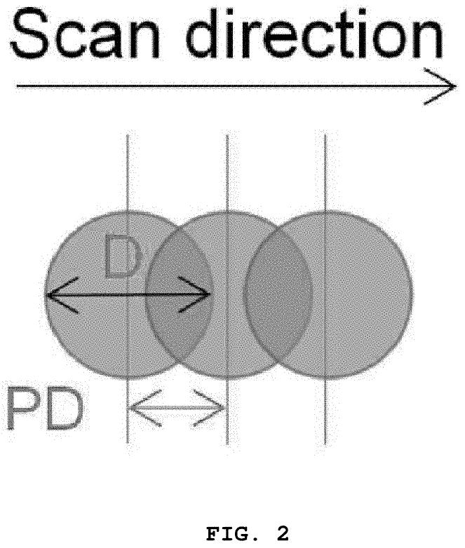

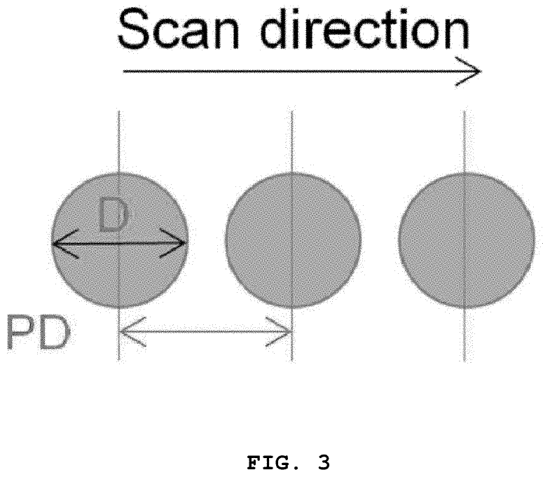

Referring to FIGS. 2 and 3, a manner of radiating a laser in the present invention can be seen. FIG. 2 shows a laser radiation manner in common laminated-molding. A laser is radiated to a base material layer in the manner shown in FIG. 2 in the present invention. The point distance PD becomes smaller than the diameter D of the laser radiation points, so the laser radiation points partially overlap one another. FIG. 3 shows a laser radiation manner when forming a porous layer in the present invention, in which the point distance PD becomes larger than the diameter D, so the laser radiation point does not overlap each other. Accordingly, metallic particles are melted only at the laser radiation points and a porous structure is formed.

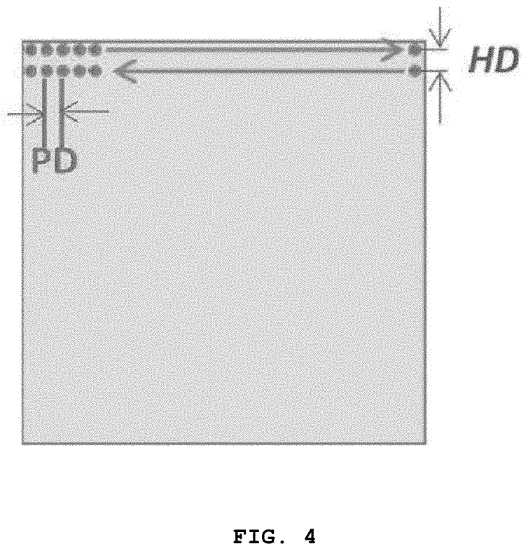

FIG. 4 shows a hatch distance and a point distance when a porous layer is formed in the present invention. It is possible to prevent laser radiation points from overlapping one another by adjusting not only the point distance, but also the hatch distance.

The diameter D of the laser radiation points may be in proportion to the source power and exposure time of the laser and the exposure time may be in inverse proportion to the scan speed of the laser.

The source power of the laser may be 50 W to 1 KW, and the scan speed may be 0.1 m/s to 8 m/s. The conditions of the source power and the scan speed may depend on the kind of metallic particles and the structure of a porous layer to be formed. For example, when a base material layer that requires high-density molding is formed using pure titanium, energy of 5.5 to 6.5 J or more per cubic millimeters should be provided, and this can be achieved in conditions of the source power of 100 W or more at a scan speed of 0.25 m/s.

Energy equal to or less than the complete melting energy can be radiated when a porous layer is formed, so the source power can be reduced at the same scan speed. Further, it is also possible to increase the scan speed with the source power maintained in order to increase the laser radiation point distance. However, when the scan speed is increased too much, the exposure time of a laser may be decreased and the diameter of the laser radiation points may become too small, so it is preferable to adjust the scan speed within the range described above.

The hatch distance and the point distance may be 100 to 1000 μm, respectively. When the point distance is less than 100 μm, the diameter D of laser radiation points that should be smaller than the point distance is too small, so machinability is deteriorated. When the point distance exceeds 1000 μm, the diameter D of laser radiation points should be correspondingly increased to be able to form a porous layer, and for this purpose, the laser source power should also be increased, so it is not preferable. Further, when the point distance exceeds 1000 μm, there is another problem that the specific surface area of the porous layer is small.

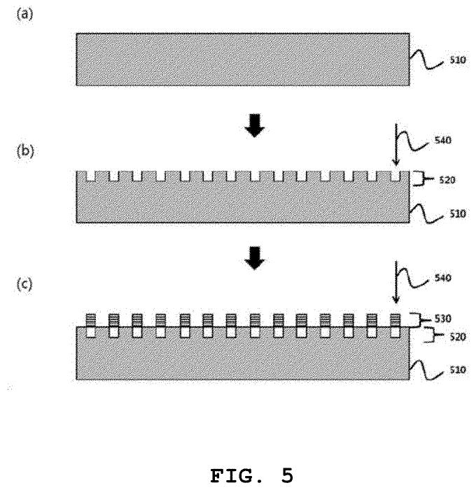

The first porous layer may be engraved. Referring to FIG. 5, a base material layer 510 is formed in (a) and then a first porous layer 520 can be formed by radiating a laser 540 in (b). The first porous layer 520 is formed by melting a portion of the base material layer 510, so it is engraved. When the laser 540 having strong source power is radiated to the surface of the base material layer 510, the surface illumination is reduced, so the first porous layer 520 is engraved.

Referring to (c) of FIG. 5, a second porous layer 530 can be formed by layering metallic particles on the first porous layer and then radiating a laser 540.

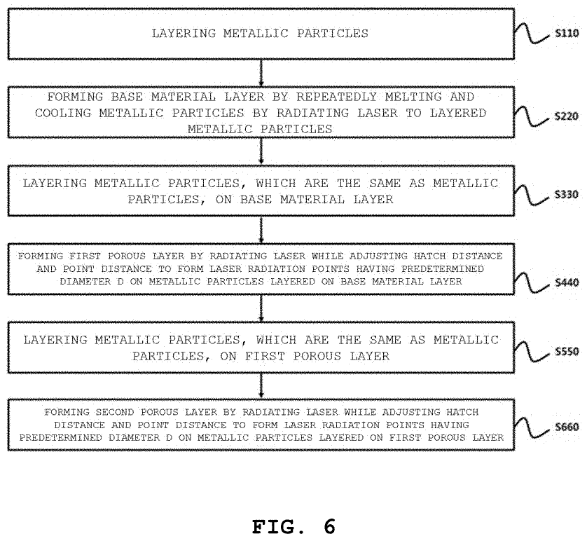

Referring to FIG. 6, an embodiment of the present invention provides a one-step manufacturing method of a laminated molding porous component, the method including the steps of: layering metallic particles (S110); forming a base material layer by repeatedly melting and cooling the metallic particles by radiating a laser to the layered metallic particles (S220); layering metallic particles, which are the same as the metallic particles, on the base material layer (S330); forming a first porous layer by radiating a laser while adjusting a hatch distance and a point distance to form laser radiation points having a predetermined diameter D on the metallic particles layered on the base material layer (S440); layering metallic particles, which are the same as the metallic particles, on the first porous layer (S550); and forming a second porous layer by radiating a laser while adjusting a hatch distance and a point distance to form laser radiation points having a predetermined diameter D on the metallic particles layered on the first porous layer (S660).

The metallic particles may be one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

In particular, titanium and titanium-based alloys, which are materials having excellent biocompatibility, have been known as having not only good biocompatibility for surrounding tissues, but large resistance against corrosion and little biological toxicity, so they are preferable. However, the present invention is not limited thereto and the metallic particles described above can be selectively used.

The laser may have energy equal to or greater than complete melting energy of the metallic particles in the step of forming the base material layer.

In the steps of forming the first porous layer and forming the second porous layer, the laser may have energy equal to or greater than 0.2 times the complete melting energy within a range equal to or less than the complete melting energy of the metallic particles.

When energy greater than the complete melting energy is applied to the metallic particles, the metallic particles may be completely melted and densified. When smaller energy is applied to the metallic particles, the metallic particles may be formed in a porous type without being densified.

That is, when forming the base material layer and the porous layers in the present invention, the base material layer can be densified by inputting energy equal to or greater than the complete melting energy and the porous layers can be formed in porous type by inputting energy equal to or greater than 0.2 times the complete melting energy within a range equal to or less than the complete melting energy. The porosity is another factor that forms a porous structure separate from radiating a laser while adjusting a hatch distance and a point distance when forming laser radiation points. When the laser has energy less than 0.2 times the complete melting energy of the metallic particles, the metallic particles are never melted, so it is not preferable.

The hatch distance and the point distance may be greater than the diameter D of the laser radiation points in the step of forming the first porous layer and the step of forming the second porous layer. Referring to FIGS. 3 and 4, it can be seen that a porous layer can be formed with the hatch distance and the point distance greater than the diameter D of the laser radiation points.

The diameter D of the laser radiation points may be in proportion to the source power and exposure time of the laser and the exposure time may be in inverse proportion to the scan speed of the laser.

The source power of the laser may be 50 W to 1 KW, and the scan speed may be 0.1 m/s to 8 m/s. The conditions of the source power and the scan speed may depend on the kind of metallic particles and the structure of a porous layer to be formed. For example, when a base material layer that requires high-density molding is formed using pure titanium, energy of 5.5 to 6.5 J or more per cubic millimeters should be provided, and the source power should be 100 W at a scan speed of 0.25 m/s.

Energy equal to or less than the complete melting energy can be radiated when a porous region is formed, so the source power can be reduced at the same scan speed. Further, it is also possible to increase the scan speed with the source power maintained in order to increase the laser radiation point distance. However, when the scan speed is increased too much, the exposure time of a laser may be decreased and the diameter of the laser radiation points may become too small, so it is preferable to adjust the scan speed within the range described above.

The hatch distance and the point distance may be 100 to 1000 μm, respectively. When the point distance is less than 100 μm, the diameter D of laser radiation points that should be smaller than the point distance is too small, so machinability is deteriorated. When the point distance exceeds 1000 μm, the diameter D of laser radiation points should be correspondingly increased to be able to form a porous layer, and for this purpose, the laser source power should also be increased, so it is not preferable. Further, when the point distance exceeds 1000 μm, there is another problem that the specific surface area of the porous layer is small.

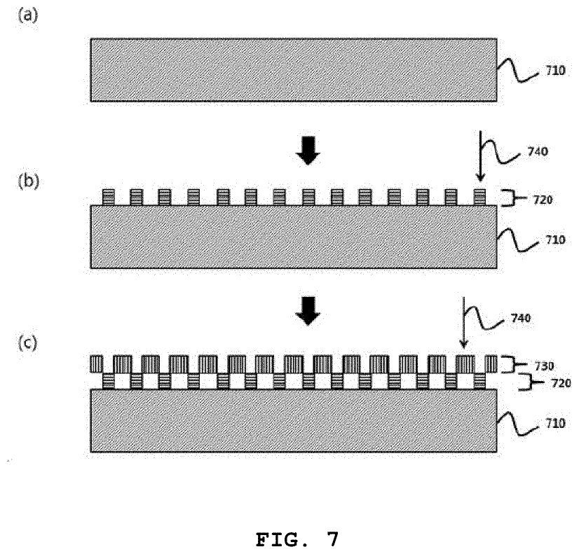

The first porous layer may be embossed. Referring to FIG. 7, a base material layer 710 is formed in (a) and then a first porous layer 720 can be formed by layering metallic particles and radiating a laser 740 in (b). Since the first porous layer 720 is formed by layering the metallic particles and then radiating a laser 740, the first porous layer 720 is embossed. In (c), a second porous layer 730 can be formed by layering metallic particles on the first porous layer and the radiating a laser.

The laser radiation points in the step of forming the second porous layer may be arranged not to overlap the laser radiation point on the first porous layer.

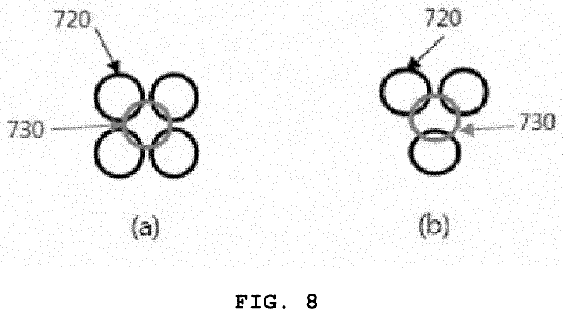

Referring to FIG. 8, the first porous layer 720 is formed in accordance with laser radiation points, and then, when the second porous layer 730 is formed on the first porous layer, the laser radiation points of the second porous layer 730 do not overlap the laser radiation points of the first porous layer 720, as in (a) or (b) of FIG. 7. Accordingly, it is possible to secure strength of the porous structure and further increase the specific surface area of the porous layer.

The present invention further provides a laminated-molding porous component that is manufactured by the method. The laminated-molding porous component according to the present invention has an integrated base material layer-porous layer, so the manufacturing time is reduced and the manufacturing process is simple in comparison to existing products formed using porous coating.

The present invention further provides an implant having an increased bone contact ratio and including the porous component. The porous component according to the present invention has many pores having a diameter of 50 to 200 μm, so it has improved bone contact ratio and bone growth in comparison to implants using a biological tissue adhesive such as bone cement. Further, since the porous layer is integrally formed, an implant that is more excellent in strength and durability can be provided.

The present invention is described in more detail hereafter with reference to a preferred embodiment. However, it should be noted that the present invention is not limited thereto and the embodiment is just an example.

### Embodiment 1

Pure titanium particles were layered and a base material layer was formed by radiating a laser at a scan speed of 0.5 m/s and source power of 200 W. A first porous layer was engraved by radiating a laser while adjusting a hatch distance and a point distance each by 350 μm to form laser radiation points having a diameter of 70 μm on the base material layer. A second porous layer was formed by layering again pure titanium particles on the first porous layer and radiating a laser while adjusting a hatch distance and a point distance each by 350 μm to form laser radiation points having a diameter of 70 μm.

### Embodiment 2

Pure titanium particles were layered and a base material layer was formed by radiating a laser at a scan speed of 0.5 m/s and source power of 200 W. A first porous layer was embossed by layering pure titanium particles on the base material layer and radiating a laser while adjusting a hatch distance and a point distance each by 350 μm to form laser radiation points having a diameter of 70 μm on the base material layer. A second porous layer was formed by layering again pure titanium particles on the first porous layer and radiating a laser while adjusting a hatch distance and a point distance each by 350 μm to form laser radiation points having a diameter of 70 μm.

The following Table 1 shows laser radiation conditions when forming the first porous layer and the second porous layer in the embodiments 1 and 2.

TABLE 1

| | Scan | Source | Exposure |
|---|---|---|---|
| | speed | power | time |
| Items | (m/s) | (W) | (μs) |

| | | | | |
|---|---|---|---|---|
| Embodiment | First porous | 0.875 | 400 | 400 |
| 1 | layer | | | |
| | Second porous | 0.875 | 400 | 400 |
| | layer | | | |
| Embodiment | First porous | 0.875 | 200 | 400 |
| 2 | layer | | | |
| | Second porous | 0.875 | 200 | 400 |
| | layer | | | |

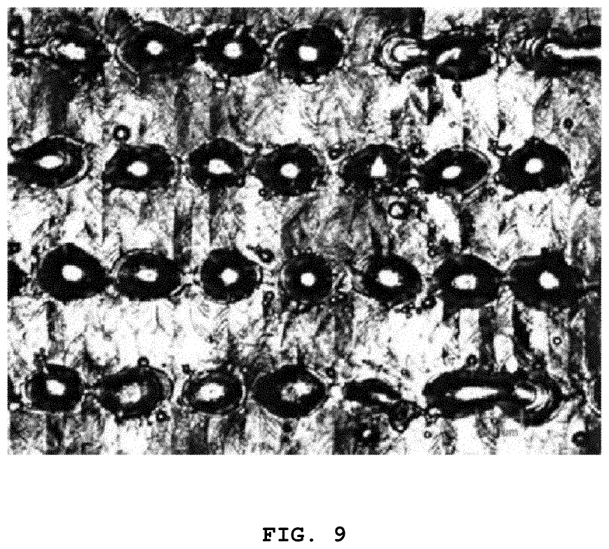

FIG. 9 is a picture of the surface of the first porous layer formed in the embodiment 2.

When a porous layer is formed in accordance with the method of manufacturing a porous component of the present invention, laser radiation conditions such as a scan speed, source power, and exposure time are set in accordance with the kind of metallic particles and the structure of a porous layer, whereby it is possible to easily design implants fitted to the frames of patients.

According to an embodiment of the present invention, it is possible to reduce a manufacturing time when manufacturing a product using one-step laminated-molding, and it is also possible to provide a porous component in which the shape and size of a porous layer can be controlled.

Further, an implant including the porous component has an increased bone contact ratio, so bone growth between bones can be improved and products fitting to the frames of individual patients can be easily designed.

The effects of the present invention are not limited thereto and it should be understood that the effects include all effects that can be inferred from the configuration of the present invention described in the following specification or claims.

The above description is provided as an exemplary embodiment of the present invention and it should be understood that the present invention may be easily modified in other various ways without changing the spirit or the necessary features of the present invention by those skilled in the art. Therefore, the embodiments described above are only examples and should not be construed as being limitative in all respects. For example, single components may be divided and separate components may be integrated.

The scope of the present invention is defined by the following claims, and all of changes and modifications obtained from the meaning and range of claims and equivalent concepts should be construed as being included in the scope of the present invention.

### REFERENCE SIGNS LIST

510, 710: base material layer
520, 720: first porous layer
530, 730: second porous layer
540, 740: laser

What is claimed is:

1. A one-step manufacturing method of a laminated molding porous component, the method comprising:
layering metallic particles;
forming a base material layer by repeatedly melting and cooling the metallic particles by radiating a laser to the layered metallic particles;
forming a first porous layer by radiating a laser while adjusting a hatch distance and a point distance to form laser radiation points having a predetermined diameter D on the base material layer;
layering metallic particles, which are the same as the metallic particles, on the first porous layer; and
forming a second porous layer by radiating a laser while adjusting a hatch distance and a point distance to form laser radiation points having a predetermined diameter D on the first porous layer.

2. The method of claim 1, wherein the metallic particles are one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

3. The method of claim 1, wherein the laser has energy equal to or greater than complete melting energy of the metallic particles in the step of forming a base material layer and in the step of forming of first porous layer.

4. The method of claim 1, wherein in the step of forming a second porous layer, the laser has energy equal to or greater than 0.2 times the complete melting energy within a range equal to or less than the complete melting energy of the metallic particles.

5. The method of claim 1, wherein the hatch distance and the point distance are greater than the diameter D of the laser radiation points in the steps of forming a first porous layer and forming a second porous layer.

6. The method of claim 5, wherein the diameter D of the laser radiation points is in proportion to source power and exposure time of the laser and the exposure time is in inverse proportion to the scan speed of the laser.

7. The method of claim 6, wherein the source power of the laser is 50 W to 1 KW and the scan speed is 0.1 m/s to 8 m/s.

8. The method of claim 5, wherein the hatch distance and the point distance are 100 to 1000 μm, respectively.

9. The method of claim 1, wherein the first porous layer is engraved.

10. A one-step manufacturing method of a laminated molding porous component, the method comprising:
layering metallic particles;
forming a base material layer by repeatedly melting and cooling the metallic particles by radiating a laser to the layered metallic particles;
layering metallic particles, which are the same as the metallic particles, on the base material layer;
forming a first porous layer by radiating a laser while adjusting a hatch distance and a point distance to form laser radiation points having a predetermined diameter D on the metallic particles layered on the base material layer;
layering metallic particles, which are the same as the metallic particles, on the first porous layer; and
forming a second porous layer by radiating a laser while adjusting a hatch distance and a point distance to form laser radiation points having a predetermined diameter D on the metallic particles layered on the first porous layer.

11. The method of claim 10, wherein the metallic particles are one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

12. The method of claim 10, wherein the laser has energy equal to or greater than complete melting energy of the metallic particles in the step of forming a base material layer.

13. The method of claim 10, wherein in the steps of forming a first porous layer and forming a second porous layer, the laser has energy equal to or greater than 0.2 times the complete melting energy within a range equal to or less than the complete melting energy of the metallic particles.

14. The method of claim 10, wherein the hatch distance and the point distance are greater than the diameter D of the laser radiation points in the steps of forming a first porous layer and forming a second porous layer.

15. The method of claim 14, wherein the diameter D of the laser radiation points is in proportion to source power and exposure time of the laser and the exposure time is in inverse proportion to the scan speed of the laser.

16. The method of claim 15, wherein the source power of the laser is 50 W to 1 KW and the scan speed is 0.1 m/s to 8 m/s.

17. The method of claim 14, wherein the hatch distance and the point distance are 100 to 1000 μm, respectively.

18. The method of claim 10, wherein the first porous layer is embossed.

19. The method of claim 10, wherein the laser radiation points in the step of forming a second porous layer are arranged not to overlap the laser radiation points on the first porous layer.

20. A laminated-molding porous component formed by the method of claim 1.

21. An implant having an increased bone contact ratio and including the porous product of claim 20.
